(19) United States
(12) Patent Application Publication
(10) Pub. No.: US 2024/0180161 A1
(43) Pub. Date: Jun. 6, 2024

(54) NANOHYBRID STRUCTURES CONTAINING ANTIMICROBIAL NANOPARTICLES

(71) Applicant: P&S Global Holdings LLC, Houston, TX (US)

(72) Inventors: Parash KALITA, Houston, TX (US); Rustom MODY, Houston, TX (US)

(73) Assignee: P&S Global Holdings LLC, Houston, TX (US)

(21) Appl. No.: 18/018,832

(22) PCT Filed: May 14, 2021
(86) PCT No.: PCT/US2021/032481

§ 371 Date: Jan. 30, 2023

Related U.S. Application Data
(60) Provisional application No. 63/058,884, filed on Jul. 30, 2020.

(51) Int. Cl. A01N 59/20; A01N 25/24; A01N 25/26; A01N 25/34; A01P 1/00; A01P 3/00

(52) U.S. Cl. CPC A01N 59/20; A01N 25/24; A01N 25/26; A01N 25/34; A01P 1/00; A01P 3/00

(57) ABSTRACT

This invention relates to forming unique nanohybrid structures through selective integration of inorganic antimicrobial nanoparticles with other inorganic and organic materials and then, chemically bonding the nanohybrid structures to organic polymers for application as surface coatings, antimicrobial surfacing, food packaging, biomedical, agricultural, air-filtration/cleaning and water filtration/cleaning applications to kill, inhibit, and/or reduce the growth of pathogenic/infectious/contaminating microorganisms and their biofilms.

### CROSS REFERENCE TO RELATED APPLICATION

The application claims the benefit of and priority to U.S. Provisional Application No. 63/058,884 entitled, “Nanohybrid Structures Containing Antimicrobial Nanoparticles” filed on Jul. 30, 2020.

### FIELD OF INVENTION

This application relates to chemicals used as coatings. More specifically, this application relates to chemically bonding nanohybrid structures to organic polymers for application as surface coatings, antimicrobial surfacing, food packaging, biomedical, agricultural, air-filtration/cleaning and water filtration/cleaning applications to kill, inhibit, and/or reduce the growth of pathogenic/infectious/contaminating microorganisms and their biofilms.

### STATEMENT REGARDING FEDERALLY SPONSORED RESEARCH OR DEVELOPMENT

Not applicable.

### REFERENCE TO A “SEQUENCE LISTING”, A TABLE, OR COMPUTER PROGRAM

Table 1 presents Ag—CaCO₃—ZnO based nanohybrid structures manufactured via chemical reduction.

Table 2 presents antimicrobial performance of polyurethane insulation foam containing organofunctionalized Ag—ZnO—CaCO₃ based nanohybrid structures.

Table 3 presents antimicrobial performance of polyamide nanohybrids containing aminofunctional Ag—Cu—SiO₂-based nanohybrid structures.

Table 4 presents AATCC 100 antimicrobial assessment of Kevlar fabrics coated with nanohybrid integrated acrylic coating

Table 5 presents nanohybrid integrated polymeric formulations for antimicrobial treatment of polyester textiles.

Table 6 presents nanohybrid integrated polymeric formulations for antimicrobial treatment of cotton textiles.

### DESCRIPTION OF THE DRAWINGS

FIG. 1 is a graphical representation of the inventive antimicrobial nanohybrid structures, in which at least one of the phases is ≤100 nm in at least one dimension.

FIG. 2 is a graphical representation of chemical bonding between organofunctionalized antimicrobial nanohybrids with organic polymers to form compatible organic/inorganic nanohybrids.

FIG. 3 presents a summary of different polymer processing techniques that can be implemented for forming polymer nanohybrids with antimicrobial nanohybrid structures.

FIG. 4 presents an example of chemical reduction-based synthesis of nanohybrid structure and TEM image of the resultant nanohybrid structure.

FIG. 5 presents an example process sequence of organosilane treatment to form organofunctionalized Ag—ZnO—CaCO₃ nanohybrid for enhanced bonding with organic polymer composition (polyurethane insulation foam).

FIG. 6 presents HAADF-STEM image of aminofunctional Ag—Cu—SiO₂ nanohybrid and EDX chemical analysis of the nanohybrid structure showing location & chemical identity of Silver (Ag) and Copper (Cu) nanoparticles.

FIG. 7 presents an illustration of mechanochemical synthesis of Ag—ZnO nanohybrid, polymeric coating formulation, and characteristics of the coated Kevlar, including SEM and EDX mapping showing the distribution of Silver-based nanohybrids in the coated Kevlar.

FIG. 8 presents scanning electron microscopy (SEM) with energy dispersive X-ray analysis (EDX) analysis of nylon 6 substrates coated with Ag—ZnO nanohybrid integrated acrylic, showing elemental distribution of Ag and Zn in the coated substrate.

FIG. 9 presents percentage reduction of viruses in cotton textiles that were treated with nanohybrid integrated acrylic-based coating (with and without C6 fluorocarbon-based hydrophobic polymer).

FIG. 10 presents percentage reduction of microbes in polyester textiles that were treated with nanohybrid integrated acrylic and hyperbranched hydrophobic (fluorocarbon-free) polymer coating.

### BACKGROUND OF THE INVENTION

Infectious microbes (including bacteria, viruses, and fungus) have tendency to contaminate surfaces by rapid multiplication to form colonies and biofilms on many different surfaces. Such surfaces are highly vulnerable to develop contagious infections and pathogens. A vast majority of today's surfaces/components across different sectors (from packaging to biomedical tubing) are made from plastics or polymers and many of such polymeric surfaces/components) cannot be physically cleaned or sanitized regularly or comprehensively. Microbial protection of such polymeric surfaces and components are very important to restrict the growth and subsequent spread of infections. Introduction of antimicrobials in appropriate chemophysical form either on the surface or in the internal structure of a polymer/plastic is an effective solution to fight this problem, which is the underlying objective of this invention.

Extensive studies has been done on the use of inorganic metal particles for imparting antimicrobial activity, such as silver, copper, zinc, brass, bronze, gold, germanium, titanium, barium, tantalum, etc. They are either used in the form of metal ions (formed when an atom gains or loses an electron) as specified in US 20030118664 and US20040224005, and as nanoparticles which constitutes an assembly of atoms. Use of inorganic metallic nanoparticles (nanosized particles predominantly of Silver and Copper based nanoparticles that has antimicrobial properties to either kill microorganism or stop their growth) for antimicrobial activity in various polymeric compositions (as nanocomposites) has been reported in publications [US20030118664, US20190345600, WO2018090152, US20200352990, CN110330678A, US20160201183, RU2704623C, CN106633927A, US20150017432, US20080051493, US20120302703]. Chemically synthesized composite particles containing metallic nanoparticles have been reported, such as Copper nanoparticles in Clay [US 20060202177A1], Copper nanoparticles in silica nanoparticles [Kim et al], copper nanoparticles in silica shell [US 20130108702] and metallic nanoparticles with terminal reactive group on its surface [U.S. Pat. No. 7,923,110]. Most studies have reported forming polymer-based nanocomposite materials by filling polymer matrix (e.g. resins or other forms) with pre-formed metallic antimicrobial nanoparticles or directly synthesizing the metallic nanoparticles in the polymer, for example by reducing metal salts to metallic nanoparticles in the polymer matrix. In general, these inorganic metallic nanoparticles are ‘free particles’ in the polymer nanocomposites, irrespective of the type of inorganic antimicrobial nanoparticles, polymeric materials, and the methodologies of manufacturing the nanoparticles or their composites with polymers. The attribution of ‘free particles’ is because inorganic metallic nanoparticles (typically hydrophilic) are immiscible with non-polar hydrocarbon-based organic polymers. Such immiscibility results in poor spatial dispersion and compatibility of nanoparticles within the polymer leading to limited or inconsistent performance.

The chemostructural properties of these nanocomposite particles/materials are significantly different from the present invention of nanohybrid structures with antimicrobial properties. Nanocomposite materials are ones that incorporate nanosized particles (for example silver or copper nanoparticles) into a matrix of a standard material (for example, clay or silica or polymers mentioned in the publications). In comparison, nanohybrids are synthetic materials with organic and inorganic constituents that are bonded together by covalent bonds or noncovalent bonds (hydrogen bond, van der Waals force or electrostatic force) at nanometer scale. The present invention involves preforming an organofunctionalized nanohybrid structure containing one or more type of inorganic antimicrobial nanoparticles deposited to one or more type of organic/inorganic material species and then, chemically (covalent) bonding the preformed nanohybrids with organic polymers to yield polymeric nanohybrids with enhanced organic-inorganic compatibility and antimicrobial activity. These unique characteristics of the novel nanohybrid structures are described below in greater detail.

### BRIEF SUMMARY OF THE INVENTION

This invention relates to forming unique nanohybrid structures through selective integration of inorganic antimicrobial nanoparticles with other inorganic and organic materials and then, chemically bonding the nanohybrid structures to organic polymers for application as surface coatings, antimicrobial surfacing, food packaging, biomedical, agricultural, air-filtration/cleaning and water filtration/cleaning applications to kill, inhibit, and/or reduce the growth of pathogenic/infectious/contaminating microorganisms and their biofilms.

### DETAILED DESCRIPTION OF THE INVENTION

In one or more embodiments, this application relates to nanohybrid structures derived through nanoscale modifications of organic/inorganic materials with antimicrobial nanoparticles and organofunctional reactive groups for better distribution of antimicrobial species, increased antimicrobial surface area and activity, and chemical compatibility/bonding while integrating the nanohybrids with organic polymers to render superior antimicrobial performance. The novel antimicrobial nanohybrid is characterized by having a composition comprising (A) one or more types of inorganic antimicrobial nanoparticles, (B) organic and/or inorganic carrier material(s) to which the antimicrobial nanoparticles are chemically and/or mechanically deposited to form organic-inorganic hybrid or entirely inorganic compositions, and (C) organosilanes for organofunctionalization of inorganic and/or hybrid compositions to form organic-inorganic nanohybrid structures. The inventive polymer nanohybrids consists of one or more types of novel antimicrobial nanohybrids in a matrix of one or more types of organic polymers. The term ‘nanohybrid’ include synthetic materials with organic and inorganic constituents/components that are bonded or linked together by covalent bonding or noncovalent bonding (hydrogen bond, van der Waals force or electrostatic force) at nanometer scale. The process of attaching nanoparticles to solid surfaces of carrier materials to create coatings of nanoparticles is herein referred to as ‘deposition’.

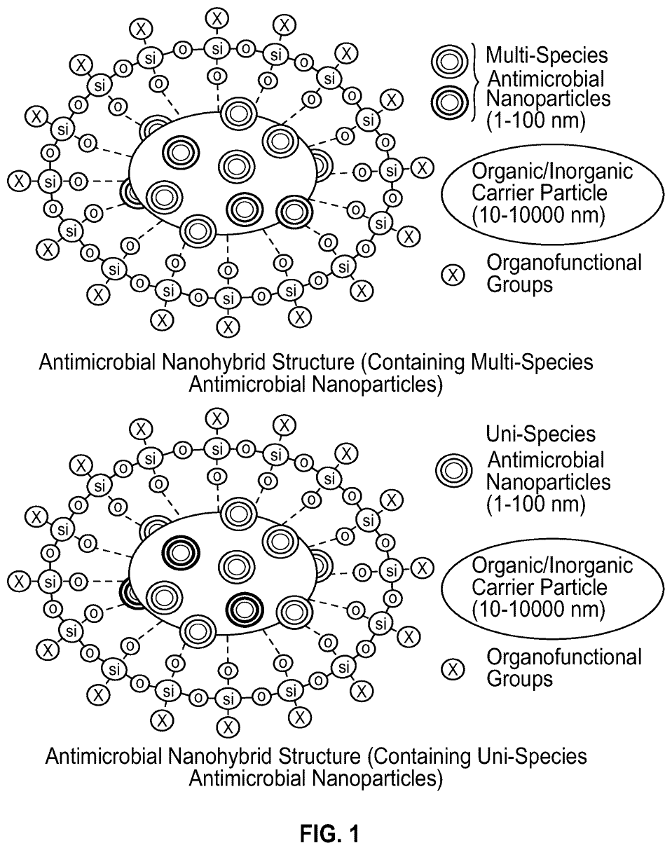

The inventive antimicrobial nanohybrid structure (as shown in FIG. 1) consist of one or more kind (chemical species) of 0.5-50 wt. % inorganic antimicrobial nanoparticles that are deposited onto inorganic and/or organic ‘carrier’ materials of 10-10000 nm in size, and the entire nanoparticles-deposited inorganic/organic structure is organofunctionalized with reactive organofunctional groups, wherein the inorganic antimicrobial nanoparticles (particles with at least one dimension ≤100 nm) include any metal-based nanoparticles that has inherent antimicrobial property to kill or stop the growth microorganisms (bacteria, fungi, and/or viruses). In the present invention, the antimicrobial nanoparticles may be selected from Silver, Copper, Copper Oxide, Zinc Oxide, Nickel, Selenium, Titanium and/or Titanium Dioxide based metallic species. The antimicrobial nanoparticles (metal and/or metal oxides) can be derived either by mechanochemical synthesis of milling large sized particles into nanosized particles (for example, milling 2.5 μm ZnO particles to <100 nm ZnO nanoparticles) or by chemical reduction of metallic salt precursors into metal/metal oxide nanoparticles (for example, reducing silver nitrate salt to silver nanoparticles or copper sulphate salt to copper oxide nanoparticles). The antimicrobial properties of the above metal nanoparticles are well known to those skilled in the art and are described in Brandelli et al, which is incorporated herein as reference.

Mechanical milling or chemical reduction is carried out in a matrix of inorganic ‘carrier’ materials to deposit the as-produced antimicrobial nanoparticles onto the inorganic ‘carrier materials’, which may include but not limited to:
Flame Retardant Materials—Metal hydroxides, Hydroxycarbonates, Borates, Inorganic phosphates, and Melamine;
Mineral Fillers: Metal carbonates/hydrogen carbonates, Aluminosilicates, and Silicate minerals;
Metal Oxides—Al₂O₃, ZnO, TiO₂, MgO, CaO, ZrO₂, Cr₂O₃, SiO₂, Copper Oxide, Ag₂O;
Transition Metallic Species—Chromium, Cobalt, Nickel, and Copper;
Biomaterials—Titanium alloys, Cobalt-Chromium alloys, Stainless Steel, and Tantalum;
Solid Lubricant Species—MoS₂, Graphite, Hexagonal Boron Nitride, PTFE, & Calcium Fluoride;
Carbon based—Graphene, Graphene Oxide, Carbon Black, Charcoal, and CNTs;
Quaternary Ammonium Compounds—Benzalkonium Chloride;
Semiconductor Materials: Silicon, silicon carbide, germanium, and gallium arsenide;
Inorganic Corrosion Inhibitors: Zinc Phosphate, Zinc Molybdate, Aluminum Phosphate;
Cosmetic Ingredients: Calcium Sodium Borosilicate, Calcium Aluminum Borosilicate.

The mechanical milling or chemical reduction is carried out in a matrix of organic ‘carrier’ materials to deposit the as-produced antimicrobial nanoparticles on the organic ‘carrier’ materials, which may include but not limited to: Carbamide Peroxide, Lysozyme, Chitosan, Starch, Collagen Peptides, Lignin, and Nanocrystalline & Nano-fibrillated Cellulose.

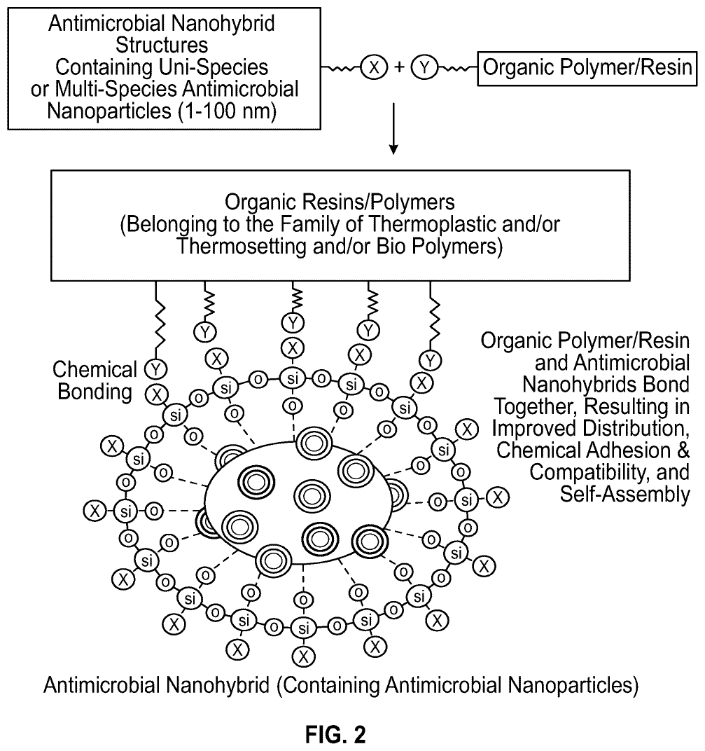

In the present invention, the antimicrobial nanohybrids consisting of the above described inorganic/organic materials are surface functionalized with organosilane coupling agents so that the nanohybrid structures contains reactive organofunctional groups. In the present invention, the organofunctional groups may include but not limited to vinyl group, epoxy group, mercapto groups, amino groups, methacryloxy group, isocyanate groups, thiol groups, methoxy groups, and ethoxy groups. Such reactive organofunctional groups can chemically bond with organic materials, and hence, facilitates the organofunctionalized antimicrobial nanohybrids to covalently bond with organic polymers/resins such as PP, PVC, nylon, LDPE, HDPE, PU, etc., as depicted in FIG. 2. Most organosilanes have one reactive organofunctional group (represented as X) and three hydrolyzable groups (represented as Y), as shown below:

X—(CH₂)ₙ—Si—Y₍₃₋ₙ₎

Where, (CH₂)ₙ refers to linker groups and Si refers to silicon present in the organosilane. During organosilane functionalization, the hydrolyzable groups (Y) bonds with the antimicrobial nanohybrid structures leaving the reactive organofunctional group available for further reaction and bonding with organic polymers.

As described in example below, the reaction of antimicrobial nanohybrids with organosilanes (e.g. aminofunctional silane) involves four steps that can occur simultaneously.

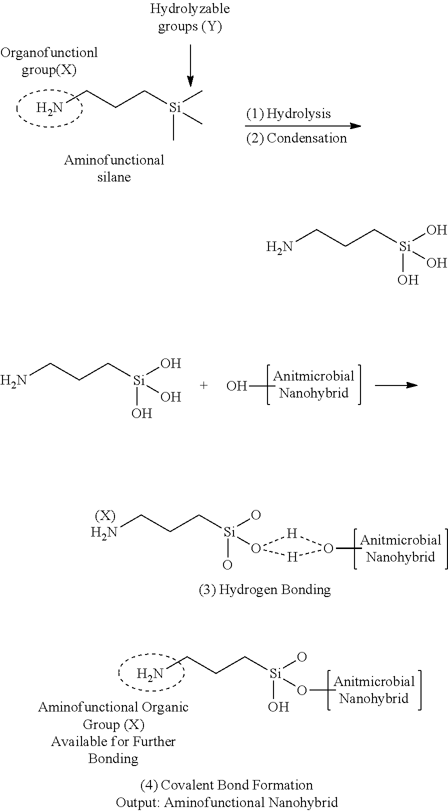

The reaction begins with the hydrolysis of the three hydrolyzable groups (Y) of organosilanes. This is followed by condensation to from oligomers and their hydrogen bonding with the surface hydroxyls of antimicrobial nanohybrids. Finally, as reaction concludes with curing, covalent linkages are formed between silicon of organosilane and antimicrobial nanohybrid surface. The organofunctional group (X) remain available for further reaction and covalent bonding with organic polymers. As a result, the novel organofunctionalized antimicrobial nanohybrids develops better chemical bridging and bonding for enhanced compatibility, adhesion, self-assembly and spatial distribution within the polymer matrix for rendering effective antimicrobial activity and durability.

The inventive antimicrobial nanohybrid structures are manufactured in two steps. Step 1 includes manufacturing of inorganic antimicrobial nanoparticles deposited to organic/inorganic carrier materials and Step 2 includes organofunctionalization of compound derived from Step 1. The first manufacturing step can be accomplished through two different processes, (a) Chemical Reduction, or (b) Mechanochemical synthesis. The selection of these two processes depends on the desired inorganic-organic hybrid composition and physical dimensions of inorganic antimicrobial nanoparticles and organic/inorganic carrier materials.

In chemical reduction method, the inorganic and/or organic carrier materials are first saturated with a solution containing metallic salt precursors (selected for desired antimicrobial nanoparticles, e.g. silver nitrate salt for silver nanoparticles, copper chloride salt for copper nanoparticles, etc.). This is followed by reacting the mixture with reducing agents together with surface-activated agents, either with or without the presence of short-chain organic alcohols at 55-135° C. under positive pressure. The reaction mixture is then dry cured under vacuum between 60-120° C. followed by dry milling into fine powder particulates. The reduction reaction followed by thermal processing yields consistent-sized antimicrobial nanoparticles (10-100 nm) deposited on the inorganic/organic material matrix. Following synthesis, the nanohybrid composition becomes surface functionalized by combining it with appropriate organosilane coupling agents.

The metallic salt precursors can be any hydrolyzable or water-soluble metallic salts which can be reduced to metal or metal oxide nanoparticles. For example, Copper/Copper oxide nanoparticles can be derived by reducing copper (II) salts including but not limited to Copper Sulphate (Cu₂SO₄), Copper Chloride (CuCl₂), Copper Hydroxide (Cu(OH)₂), Copper Nitrate (Cu(NO₃)₂, Copper Fluoride (CuF₂), Copper Acetate (Cu(OAc)₂), Copper Bromide (CuBr₂), Copper Formate (C₂H₂CuO₄), Copper Phosphate (Cu₃(PO₄)₂n(H₂O)), Copper Chromite (Cu₂Cr₂O₅), Copper Hexafluorosilicate (CuF₆Si), and/or Copper Selenate (CuO₄Se). Silver nanoparticles can be derived by reducing silver-based salts including but not limited to Silver Nitrate (AgNO₃), Silver Fluoride (AgF₂), Silver Nitrite (AgNO₂), Silver Perchlorate (AgClO₄), Silver Carbonate (AgCO₃), Silver Chloride (AgCl₂), etc. Similarly, zinc oxide and titanium oxide nanoparticles can be derived from their salts, such as zinc nitrate and titanium tetrachloride, respectively.

Surface active agents can be any surfactant or dispersant containing cationic, anionic, non-ionic, and zwitterionic groups or the combination of any two of the functional groups in one molecule. Examples of such surface active agents are but not limited to cyclodextrin, poly(vinyl pyrrolidone), poly(ethylene glycol), poly(vinyl alcohol), sodium dodecyl benzenesulfonate, abietic acid, polyethoxylated octyl phenol, sorbitan monoester, glycerol diester, dodecyl betaine, N-dodecyl pyridinium chloride, sulfosuccinate, 2-bis(ethyl-hexyl) sodium sulfosuccinate, alkyl dimethyl benzyl-ammonium chloride, cetyl trimethyl ammonium bromide, and hexadecyl trimethyl ammonium bromide; preferred surface active agents are cyclodextrin, poly(ethylene glycol), poly(vinyl pyrrolidone), poly(vinyl alcohol), sorbitan momoester, glycol diester; and the most preferred are poly(ethylene glycol) and poly(vinyl pyrrolidone). During chemical reduction, water and alcohol ratio is optional but preferred use not more than 70% of the alcohol in the aqueous mixture.

The reducing agent can be the compounds containing acidic or basic groups, or pH neutral groups. As used herein, a reducing agent is any substance that tends to bring about reduction by being oxidized and losing electrons. Examples of the reducing agents include citric acid, boric acid, hydrazine monohydrate, butyl aldehyde, diethylene glycolmonobutyl ether, sodium boric acid, sodium citrate, ascorbic acidcetyltrimethyl ammonium bromide, ammonia, sodium hydroxide, hydrogen peroxide, and/or hydroxyl benzaldehyde.

Mechanochemical synthesis like high-energy mechanical/ball milling is a nanomanufacturing method in which mechanical and chemical phenomena are coupled on a molecular scale to from nanosized particles and as well as composite and/or hybrid particles with uniform grain sizes and complex compositions. In mechanochemical synthesis, the reactants—inorganic antimicrobial particles (e.g. Silver and/or copper oxide) and organic/inorganic carrier materials (e.g. ZnO, Carbamide Peroxide), and optional auxiliary additives are fed in appropriate size ratios and concentrations to a high-energy mill (attritor or ball mill) loaded with milling media (ceramic or hardened steel balls). The reactants are ball milled for specific periods to produce structures with desired compositional and morphological characteristics. The expanded movement of media at high RPMs exerts various forces such as impact, rotational, shear, and tumbling leading to repeated fracturing, cold welding, amorphization, and rewelding of blended particles to yield a homogeneous compound from dissimilar materials (e.g. a composition of Silver-ZnO-Carbamide Peroxide) and at the same time, size reductions and shape modifications as a function of milling time and ratio of milling media to reactants. For manufacturing the inventive nanohybrids, mechanochemical synthesis can be performed in two ways—direct milling/grinding involving only the reactants (antimicrobial and carrier materials) and other in the presence of auxiliary additives (usually liquids and/or ions) with the reactants. The later can significantly increase the activity of the reactants for thorough and easy reactions. The auxiliary additives may be selected from but not limited to water (H₂O), salts (sodium chloride, potassium dichromate, potassium nitrate, copper sulphate and alkali metal salts) and/or organic solvents (methanol, ethanol, propylene glycol, propanol, cyclohexane, benzene, toluene, cyclohexanone, ethers and chlorinated solvents). Examples of organic solvents are listed in Joshi et al, which is incorporated herein as reference. Following mechanochemical synthesis, the nanohybrid composition becomes surface functionalized by combining it with appropriate organosilane coupling agents.

The silane functionalization of the antimicrobial nanohybrids can be accomplished by any of several methods known to those skilled in the art, such as, but not limited to reactive mixing treatment, anhydrous liquid phase deposition and vapor phase deposition. Reactive mixing treatment is presented here as a preferred method. The process involves mixing an appropriate organosilane in the form of a concentrate (typically, 0.5-1 wt. % of nanohybrid weight) or a hydrolyzed solution (typically 0.5-2 wt. %) with nanohybrid structures (in dry condition or wet state in the presence of a compatible a solvent solution) at room temperature. This is followed by filtering out and/or heat assisted dry curing (˜100-150° C.) of the excess solution to yield organofunctionalized nanohybrid structures. Reactive mixing treatment can simultaneously execute the necessary steps of hydrolysis, condensation, hydrogen bonding, and covalent bonding to yield organofunctionalized antimicrobial nanohybrid structures.

A variety of silane coupling agents are commercially available for organofunctionalization of the antimicrobial nanohybrids. They may be selected from the following based on criteria of physical dimension of the substrates, number and type of surface hydroxyl groups on the substrates (substrates in this case are the nanohybrid structures):
Vinyl functional group—vinyltrimethoxysilane, vinyltriethoxysilane
Amino functional group—3-Aminopropyltrimethoxysilane, 3-Aminopropyltriethoxysilane, 2-Dimethoxy-1,6-Diaza-2-Silacyclooctane, N-(2-Aminoethyl)-2,2,4-Trimethyl-1-Aza-2-Silacyclopentane, N-(3-Aminopropyldimethylsilyl)Aza-2,2-Dimethyl-2-Silacyclopentane
Mercapto functional group—3-Mercaptopropylmethyldimethoxysilane, 3-Mercaptopropyltrimethoxysilane
Epoxy functional group—2-(3,4 epoxycyclohexyl) ethyltrimethoxysilane, 3-Glycidoxypropyl methyldimethoxysilane, 3-Glycidoxypropyl methyldiethoxysilane.
Isocyanate functional group—3-Isocyanatopropyltriethoxysilane
Acryloxy functional group—3-Acryloxypropyl trimethoxysilane
Methacryloxy groups—3-Methacryloxypropyl triethoxysilane, 3-Methacryloxypropyl methyldimethoxysilane, 3-Methacryloxypropyl methyldiethoxysilane
Ureide group—3-Ureidopropyltrialkoxysilane

The inventive nanohybrid structures carry antimicrobial characteristics to kill and/or resist growth and propagation of a broad spectrum of pathogenic and infectious microbes, including but not limited to bacteria, fungi, and viruses. The term ‘bacteria’ include, but not limited to gram-positive and gram-negative bacteria. Examples of such bacteria species are listed in US20130108702A1, which is incorporated herein as reference. The term ‘fungi’ as used herein includes, but not limited to yeasts, rusts, smuts, mildews, molds, and include species in the, Aspergillus, Acremonium, Penicillium, Cladosporium, Ophiostoma, Magnaporthe, Fusarium, Mucor, Nerospora, Rhizopus, Tricophyton, Uredinalis, Botryotinia, Phytophthora, and Stachybotrys genera. The term ‘virus’ includes, but not limited to airborne and direct contact viruses such as Rhinoviruses, Influenza viruses, Human coronavirus, Varicella viruses, Measles virus, Hantavirus, Viral meningitis, SARS virus, etc.

In another embodiment of disclosure, this invention relates to forming unique polymer nanohybrids by incorporating, bonding, and reinforcing organic polymers with above described organofunctionalized antimicrobial nanohybrids. The inventive polymer nanohybrids consist of an organic polymer and/or copolymer as continuous phase or matrix containing dis-continuous or dispersed phase of antimicrobial nanohybrid structures. Incorporation of antimicrobial nanohybrids (e.g. organofunctionalized Ag—ZnO nanohybrid) will impart antimicrobial characteristics to the polymer composition (e.g. polypropylene) and end-use products manufactured from such polymers (e.g. HEPA filter fibers). Therefore, the polymer nanohybrids are also referred as antimicrobial polymer nanohybrids. In addition to antimicrobial properties, the incorporation of antimicrobial nanohybrids structures may also strengthen/reinforce the polymer matrix by introducing unique properties, such as mechanical strength, toughness and electrical or thermal controlled properties. The inventive polymer nanohybrids can also be formed from both organofunctionalized antimicrobial nanohybrids and as well as from non-functionalized ones. Although, the former is preferred due to covalent bonding of organofunctional groups with organic polymer networks for enhanced organic-inorganic compatibility and antimicrobial activity.

In one embodiment, the antimicrobial polymer nanohybrids consists of 20 wt. % to 99 wt. % of an organic polymer/copolymer composition and 1 wt. % to 80 wt. % of antimicrobial nanohybrid(s), wherein organic polymer/copolymer materials can be selected from: (a) Thermoplastics—HDPE, LDPE, LLDPE, Polypropylene, Acrylic, Polyamide nylon (6, 66, 6/6-6, 6/9, 6/10, 6/12, 11 & 12), polycarbonate, polystyrene, ABS, PVC, Teflon, Polyester, and PAA; (b) Thermosetting—Epoxy, phenolic, vinyl ester, polyurethane, fluoropolymers, cyanate ester, poly ester, urea formaldehyde, and silicone/polysiloxane; and/or (c) Biopolymers derived from isoprene polymers, natural polyphenolic polymers, cellulose/nano cellulose, lignin, melanin, and/or complex polymers of long-chain fatty acids.

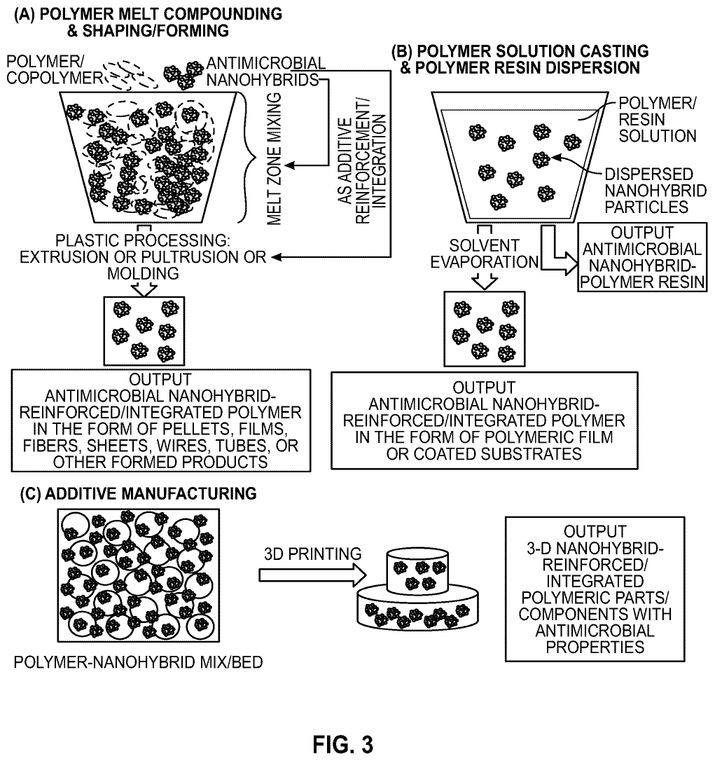

To accommodate different applications, the polymer nanohybrids can be manufactured from thermoplastic polymers, thermosetting polymers, and biopolymers as continuous phase or matrix, and the antimicrobial nanohybrid(s) incorporation/reinforcement process can be accomplished in solid, semi-solid, and liquid phase of matrix polymers. Various plastic/polymer processing techniques can used for manufacturing the inventive polymer nanohybrids with the antimicrobial nanohybrid structures depending on the quantity and production rate, dimensional accuracy and surface finish, form and detail of the product, nature of polymeric material and size of final product. The incorporation of the antimicrobial nanohybrids in polymers to form polymer nanohybrids can be accomplished by the following processing techniques as shown in FIG. 3: Polymer compounding or melt blending, shaping or forming, polymer solution casting, and additive manufacturing.

Polymer compounding or melt blending involves mixing and/or blending organic polymers/copolymer resins with antimicrobial nanohybrids and other additives/fillers relevant for the polymeric products such as coloring pigments, reinforcing materials, antioxidants, UV stabilizer, plasticizers, antistatic agents, etc. The compounded polymer-antimicrobial nanohybrid blend can be fed directly or can be converted into solid pellets, composite resins and blends before feeding to shaping/forming processes described below.

The compounded material mix can be processed through different industrially available shaping or forming techniques, including but not limited to Thermoforming, Compression and transfer molding, Rotational molding and sintering, Extrusion and extrusion-based processes, Injection molding, Blow molding and/or Plastic foam molding. All these processes utilize some kind of constraint followed by cooling/curing to form antimicrobial polymer nanohybrids in desired shape and size configurations (such as films, tubes, fibers, sheets, and other configurations).

Polymer solution casting is a processing technique where the antimicrobial nanohybrid structures are thoroughly mixed and dispersed (using powder dispersion, solution mixing and/or wet milling/grinding procedures) in organic polymers dissolved or dispersed in a solution. The mixed solution is coated onto a carrier substrate, and then the water or solvent is removed by drying to create a solid layer on the substrate. The resulting cast layer can be left as an antimicrobial coating overlayer or can be stripped from the carrier substrate to produce a standalone antimicrobial nanohybrid film.

The manufacturing of the inventive antimicrobial polymer nanohybrids is extended to additive manufacturing (also known as 3D printing) techniques as well. In AM processes, a polymer composite or a powder bed consisting of well-homogenized antimicrobial nanohybrid structures in a polymer matrix is deposited layer upon layer into precise geometric shapes. Computer-aided-design (CAD) software or 3D object scanners directs the AM hardware that consists of a heat or high energy power source (e.g. laser, thermal print head) to consolidate material (nanohybrid-polymer mix or complex) through layering method to form 3D objects with antimicrobial properties.

The above processing techniques facilitates the dispersion and bonding of antimicrobial nanohybrid structures within the polymer matrix, including covalent bonding between organofunctional groups and organic polymer networks and forming/shaping polymeric parts/products with desired configuration and antimicrobial properties for industrial and consumer use. An example of covalent bonding between a thermoset urethane polymer and amino functionalized nanohybrid structure during polymer processing is given below.

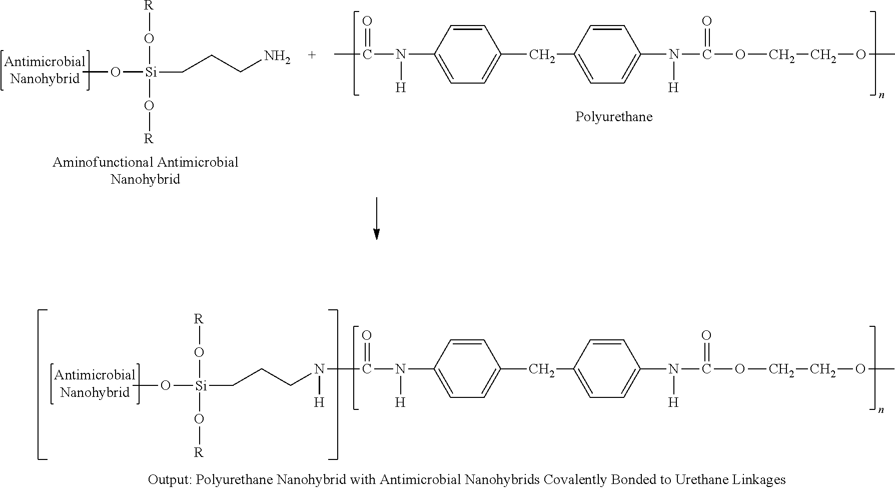

The application claims of the antimicrobial nanohybrids and nanohybrid-polymers are extended to composites, surface coatings & treatments, antimicrobial surfacing, food packaging (contact and non-contact), biomedical, agricultural, water-filtration/purification, air-filtration/purification, and self-cleaning biocidal-organic/liquid repellent surface applications involving antimicrobial activity against gram-positive and gram-negative bacteria, fungi, and virus species.

Based on the results of performance testing set forth below, the inventive examples have been demonstrated to represent a new class of nanohybrid structures capable of superior antimicrobial performance.

The following examples are given for the purpose of illustrating the invention and are not intended to limit the invention. All percentages and parts are based on weight unless otherwise indicated.

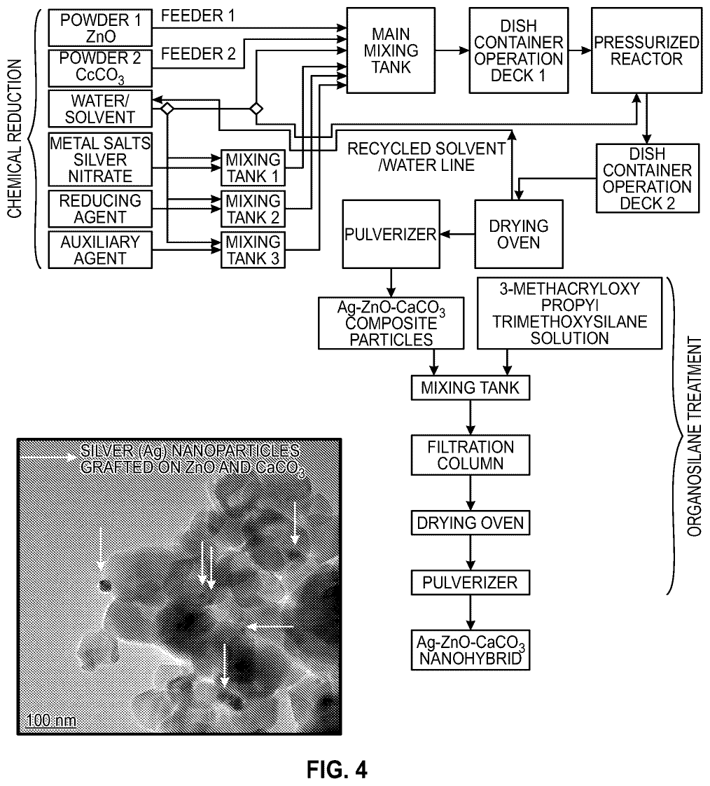

Example 1: Ag—CaCO₃ and Ag—CaCO₃—ZnO nanohybrid structures derived from chemical reduction and organofunctionalization. In Example 1, Ag—CaCO₃ and Ag—CaCO₃—ZnO structures were first manufactured via chemical reduction method, as listed in Table 1. As shown in FIG. 4, the process involved saturating the inorganic carrier materials (commercially available ZnO and CaCO₃ particulates) with Silver Nitrate (as metallic salt precursor) in an aqueous alcohol solution. The saturated aqueous mixture was then reacted with hydrazine monohydrate as reducing agent for reduction of Silver Nitrate to Silver (Ag) nanoparticles deposited in the matrix of CaCO₃ (010-701) and ZnO—CaCO₃ (010-701 & 010-703). As shown in the TEM image of the nanohybrid structure in FIG. 4, spherical-shaped silver nanoparticles (˜15-20 nm) can be seen deposited on the inorganic carrier particles. As mentioned in the description of invention, the metallic salts (nanoparticles precursors) can be any hydrolyzable or water-soluble metal salts which can be reduced to desired species of metallic nanoparticles with antimicrobial properties.

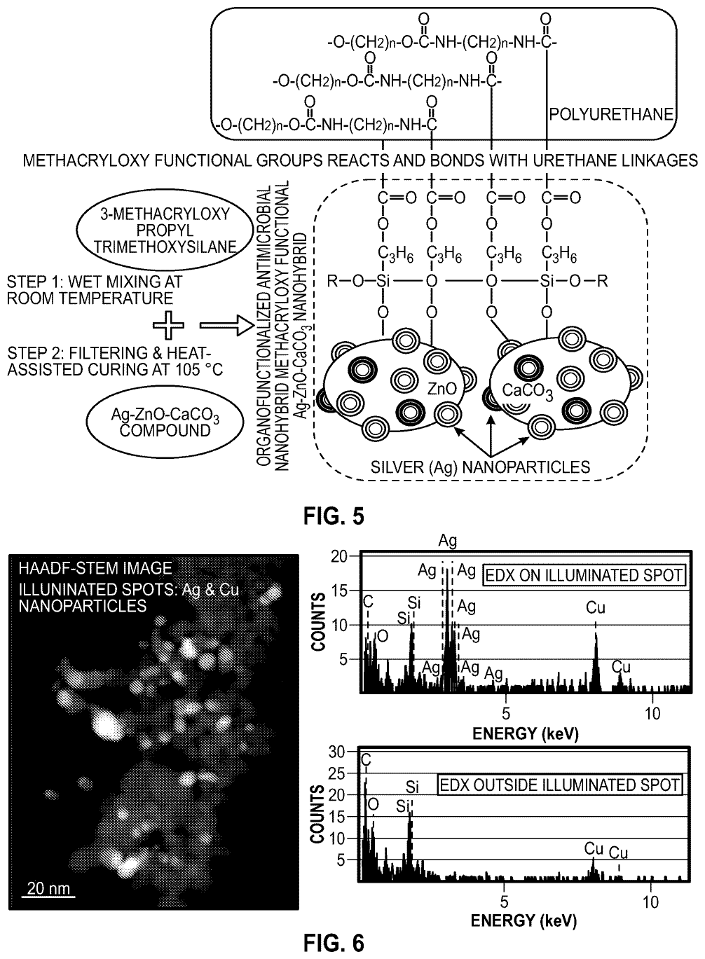

As next step of nanohybrid manufacturing, the 010-701, 010-702, and 010-703 compositions (as obtained from chemical reduction step) were organofunctionalized using a commercially available 3-Methacryloxypropyl trimethoxysilane to generate methacryloxy functionalized nanohybrid structures, as depicted in FIGS. 4 and 5. 3-Methacryloxypropyl trimethoxysilane is a di-functional organosilane having a reactive acrylic group (to bond with organic polymers) and three hydrolyzable methoxy groups (to bond with the nanohybrids), thereby acting as an interphase bridge to bond antimicrobial nanohybrids with organic polymers as shown below.

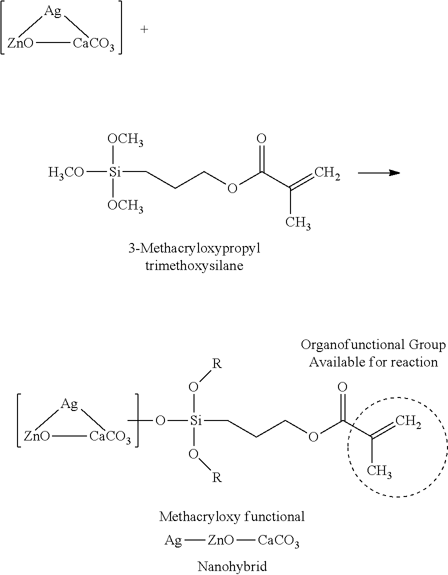

To obtain methacryloxy functionalized nanohybrid structures, the compositions obtained from chemical reduction step were mixed with a 2.0 wt. % solution of 3-Methacryloxypropyl trimethoxysilane in a 50-50 mix of DI water and methanol. This was followed by filtering out the excess solvent and curing the mixture at 105° C. until it completely dried. The dried compound was then pulverized in a low-powered hammer mill to obtain fine particulates of organofunctionalized nanohybrid structures.

Example 2: Application of antimicrobial nanohybrids in polymeric thermal insulation foam. Like household and industrial buildings, spray-applied polyurethane insulation foams and sealants have been used in livestock operations as well, for example, chicken/poultry farms, for the purpose of energy saving and improvement of envelope of the building. Such polyurethane foams and sealants can become the carriers of bacteria (such as Salmonella enterica) for spreading infectious livestock diseases and also, a source of human foodborne infection. To address this issue, the inventive antimicrobial nanohybrid structures were first incorporated into a formulated acrylic polyol and then, mixed with isocyanates. The mixture was sprayed onto the interior walls of poultry farm buildings and upon polyol-isocyanate reaction, urethane networks were formed to yield a porous polyurethane insulation foam sealant. Organofunctionalized Ag—CaCO₃—ZnO nanohybrids (010-702 and 010-703 compositions as described in Example 1) were used in this application. During polyol and isocyanates reaction, the methacryloxy functional groups of organofunctionalized Ag—CaCO₃—ZnO nanohybrids formed covalent linkages with the resultant polyurethane as shown below.

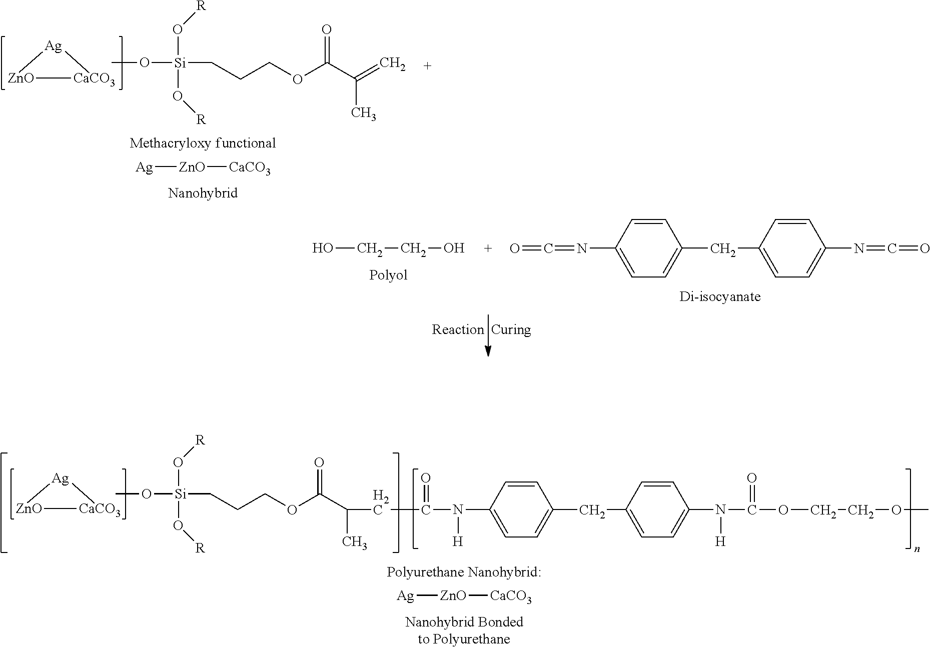

Improved bond strength from covalent bonding ensured strong adhesion between the antimicrobial nanohybrids and the resultant polyurethane matrix for durable performance. This was tested by pressure washing the nanohybrid-integrated polyurethane foams multiple times before exposing them to bacteria for antimicrobial testing as per JIS Z2801 standard. The strongly bonded nanohybrid polyurethane system resisted leaching from pressurized water impact to produce effective antimicrobial performance as listed in Table 2. The antimicrobial effectiveness was measured from the growth reduction of microbial colony forming units.

% CFU growth reduction=[1−(A/C)]×100

Where, A and C refers to the number of microbial colony forming units recovered from the treated sample after the 24 h contact period and untreated/control test sample prior to application, respectively.

As shown in Table 2, 010-702 and 010-703 nanohybrid incorporated polyurethane foam sealants reduced the growth of Salmonella enterica on insulation foam surfaces by up to 97%, as compared to the control polyurethane foams. Such reduction in bacterial count is highly favorable to the industry for maintaining healthy poultry/livestock.

Example 3: Nanohybrid Ag—Cu—SiO₂/Polyamide. Ag—Cu—SiO₂ antimicrobial nanohybrid structure was manufactured by via chemical reduction method by reducing and depositing 1.5 wt. % Silver and 1.5 wt. % Copper nanoparticles (5-10 nm) from their salt precursors (Silver nitrate and Copper (II) sulfate) on inorganic silicon dioxide (SiO₂) particles ranging between 25-100 nm in size. The resultant Ag—Cu—SiO₂ composition was then treated with an Aminofunctional silane coupling agent (3-Aminopropyltrimethoxysilane). As shown below, the objective of organosilane treatment was to generate organofunctional amine (—NH₂) groups for improved bonding and compatibility of antimicrobial nanohybrids with polyamides (an organic polymer belonging to the family of thermoplastic polymers).

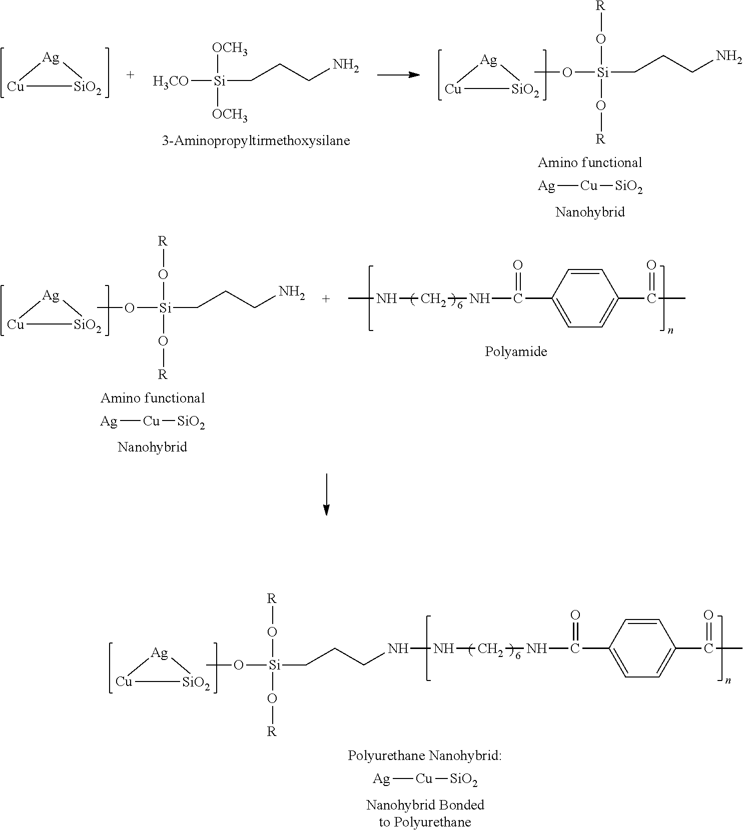

To confirm the deposition of Ag and Cu nanoparticles on inorganic silicon dioxide, high-angle annular dark-field scanning transmission electron microscopy (HAADF-STEM) was performed on the nanohybrid structure as shown in FIG. 6. HAADF-STEM confirmed the deposition of Ag and Cu nanoparticles (as illuminated spots) on inorganic silicon dioxide particles. The location and chemical identity of nanoparticles was further confirmed from Energy dispersive X-ray analysis (EDX). As shown in FIG. 6, EDX analysis confirmed elemental composition of Silver (from Ag nanoparticles), Copper (from Cu nanoparticles) and Silicon (from organosilane) on the illuminated spots. While EDX analysis on the non-illuminated spots returned elemental signature of only silicon (Si) derived from organosilane and inorganic silicon dioxide.

The polyamide nanohybrids were manufactured by dispersing the Aminofunctional nanohybrid structures (5.0 wt. %) in a solvent-borne polyamide binder and then, forming thin composite films via polymer solution casting method. During this process, the Aminofunctional groups form covalent linkages with polyamide to form a strongly bonded network of antimicrobial nanohybrids within the composite polymer films. In addition to composite films, such polyamide nanohybrids can also be utilized for manufacturing fibers and their end products such as woven/non-woven articles, biomedical tubing, and coatings with antimicrobial characteristics. As shown in Table 3, the polymeric films derived from nanohybrid integrated polyamide yielded excellent antimicrobial performance by inhibiting the colonialization of both gram positive and gram-negative microbes by 99.99%. Such antimicrobial performance is very encouraging to expand the use of such polymer nanohybrids for manufacturing fibers and their end products such as woven/non-woven articles, biomedical tubing, and coatings with antimicrobial characteristics.

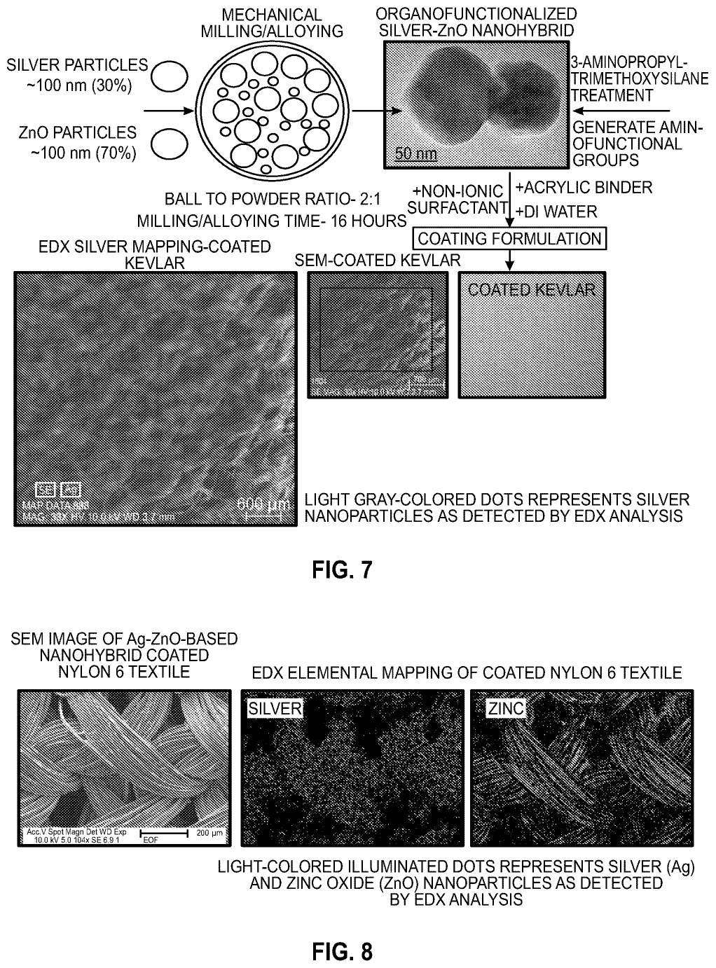

Example 4: Application of Ag—ZnO Nanohybrid-Integrated Polymer Coating Treatment. For this application, mechanochemical synthesis was used for manufacturing Ag—ZnO based nanohybrid as summarized in FIG. 7. Mechanochemical synthesis was accomplished by feeding commercially available inorganic nanoparticles (≤100 nm) of silver (2.0 wt. %) and zinc oxide particles (98.0 wt. %) to high-energy ball milling apparatus for a period of 16 hours with 1:2 powder to milling media ratio. During high-energy ball milling, the expanded movement of milling media at high RPMs resulted in various forces such as impact, rotational, shear, and tumbling leading to repeated fracturing, cold welding, amorphization, and rewelding of blended particles to yield a homogeneous Ag—ZnO compound from dissimilar materials. The composition was then treated with 3-Aminopropyltrimethoxysilane (a commercially available Aminofunctional silane coupling agent) to generate Aminofunctional Ag—ZnO nanohybrid structures. Also shown in FIG. 7, a coating solution was formulated by adding the resultant Aminofunctional nanohybrids in an aqueous solution containing thermoreactive acrylic-based polymer binder and surfactant chemistries. The coating was applied to a Kevlar (para-aramid aromatic-polyamide based) ballistic textile using pad-cure chemical finishing process. To analyze the distribution of antimicrobial nanohybrids, Scanning Electron Microscopy (SEM) with Energy dispersive X-ray analysis (EDX) analysis was performed on the coated Kevlar fabrics as shown in FIG. 7. The analysis confirmed consistent distribution of silver-based nanohybrid structures as shown by the EDX-assisted detection of elemental silver (as light gray-colored dots) in the coated Kevlar. Antimicrobial performance of the coated Kevlar was tested and compared against control Kevlar (no coating) and a commercial silver knitted Kevlar. Table 4 shows the results of antimicrobial testing performed on Kevlar textiles as per AATCC 100 method. Antimicrobial performances were measured against S. epidermidis and Escherichia coli microbes. As per measurements, Ag—ZnO nanohybrid/acrylic coated Kevlar showed >99% microbial reduction as compared to both control (untreated/uncoated) and commercial silver-knitted Kevlar.

Example 5: Fungistatic Application of Ag—ZnO Nanohybrid-Integrated Polymeric Treatment. The polymeric formulation containing thermoreactive acrylic binder and Aminofunctional Silver (Ag)-Zinc Oxide (ZnO) nanohybrid described in example 4 was used for treating 500D nylon 6 based textile substrates. FIG. 8 shows Scanning Electron Microscopy (SEM) with Energy dispersive X-ray analysis (EDX) analysis of the nanohybrid coated nylon 6 textile. The analysis confirmed consistent distribution of silver (Ag) and zinc oxide (ZnO) derived from Ag—ZnO nanohybrid structures as shown by the EDX-assisted detection of elemental silver and zinc oxide (as light gray-colored dots) in the coated substrates. Fungistatic properties of the treated substrates were measured using AATCC 30 method-Antifungal Activity, Assessment on Textile Materials: Mildew and Rot Resistance of Textile Materials. AATCC 30 Test III in agar plate with Penicillium variable (4.2×10⁶ CFU/ml) and Aspergillus niger (2.5×10⁶ CFU/ml) fungal species showed no growth on treated substrates after 7 and 14 days of inoculation. AATCC 30 Test IV in humidity jar (moisture chamber) with spore mix of Penicillium variable and Aspergillus niger showed no growth on treated substrates after 28 days of inoculation, showing the effectiveness of the nanohybrid polymeric treatment in controlling the growth of mold and fungi in outdoor environment.

Example 6: Application of Ag—ZnO-Carbamide Peroxide Nanohybrid-Integrated Polymeric Finish Treatment in Protective Healthcare Textiles. A variety of textile materials are used in medical and related healthcare and hygiene sectors. Their application ranges from the simple cleaning to the advanced barrier fabrics and protective garments. It is essential that such textile products are clean, and a strict control of infection is maintained. With rapid increase in blood-borne diseases, many medical textiles and garments needs barrier/resistance to fluids, such as water, blood, etc. A nanohybrid composition based on Ag—ZnO-Carbamide Peroxide was manufactured for integration with fluoropolymers and hyperbranched polymers for providing combinatorial antimicrobial and fluid repellency to polyester and cotton-based textiles specifically designed for healthcare applications. The nanohybrid was manufactured by chemically reducing silver nitrate salt to metallic silver (Ag) nanoparticles (1.0 wt. %) and depositing them in a hybrid organic-inorganic matrix consisting of 89 wt. % of Zinc Oxide (ZnO) and 10 wt. % of Carbamide Peroxide (CH₆N₂O₃). The resultant compound was treated with a commercially available Aminofunctional silane coupling agent (3-Aminopropyltrimethoxysilane) to generate Aminofunctionalized Ag—ZnO-Carbamide Peroxide nanohybrid. Treatment formulations were synthesized by dispersing 2 wt. % of Aminofunctionalized nanohybrids in a fluoropolymer and hyperbranched polymeric emulsions containing a heat-reactive acrylic copolymer, as listed in Table 5 and 6. The treatment was applied to polyester and cotton-based textiles using pad-cure chemical finishing process. In this chemical finishing process, the polyester and cotton textiles were impregnated with treatment formulations using a padding and mangle technique followed by a thermal curing step to fix and crosslink the polymeric treatment containing antimicrobial nanohybrids to the fabrics. During the thermal fixation step, the Aminofunctional groups reacts and bond to the polymers for developing a well-distributed and durable network of antimicrobial nanohybrids in the treated textile fabrics.

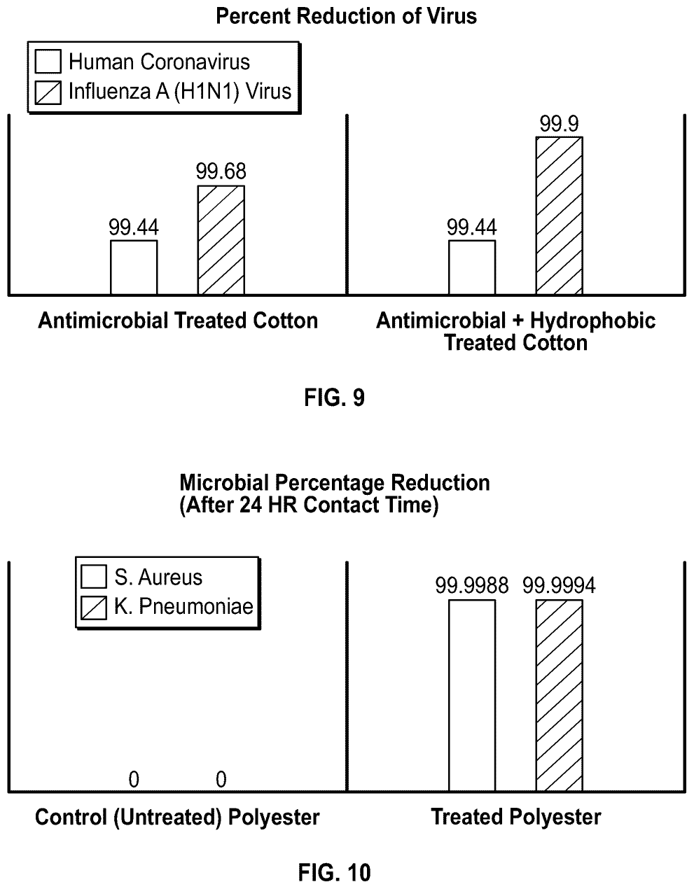

Virucidal efficacy of treated cotton textiles was carried out as per AATCC-100 test method for antibacterial finishes on textile materials modified for viruses. Influenza A (H1N1) virus strain and Human Coronavirus strain (strain 229E) were used for 24-hour contact testing at ambient room temperature. An excellent percentage reduction of over 99% in virus population was measured with the inventive nanohybrid treated cotton textiles as shown in FIG. 9.

Antimicrobial efficacy testing of treated polyester textiles was carried out as per AATCC-100 test method for antibacterial finishes on textile materials. The tests were conducted with Staphylococcus Aureus (gram positive human flora bacterium) and Klebsiella pneumoniae, a gram-negative bacterium that can cause different types of healthcare-associated infections, including pneumonia, bloodstream infections, wound or surgical site infections, and meningitis. The nanohybrid treated polyester textiles measured an impressive 99.999% reduction (log reduction of 5) against both Staphylococcus Aureus and Klebsiella Pneumoniae microbes, as shown in FIG. 10.

### REFERENCES

1. US 20030118664
2. US20040224005
3. US20190345600
4. WO2018090152
5. US20200352990
6. CN110330678A
7. US20160201183
8. RU2704623C
9. CN106633927A
10. US20150017432
11. US20080051493
12. US20120302703
13. US20060202177A1
14. Y. H. Kim, D. K. Lee, H. G. Cha, C. W. Kim, Y. C. Kang, Y. S. Kang, Preparation and Characterization of the Antibacterial Cu Nanoparticle Formed on the Surface of SiO² Nanoparticles, The Journal of Physical Chemistry B 110, 24923-24928 (2006).
15. US20130108702A1
16. U.S. Pat. No. 7,923,110
17. Brandelli, Adriano & Ritter, Ana & Veras, Flávio. (2017). Antimicrobial Activities of Metal Nanoparticles. 10.1007/978-3-319-63790-7_15.
18. Joshi, Dirgha & Adhikari, Nisha. (2019). An Overview on Common Organic Solvents and Their Toxicity. Journal of Pharmaceutical Research International. 1-18.

TABLE 1

| Composition | 010-701 | 010-702 | 010-703 |
|---|---|---|---|
| Powder 1: Inorganic CaCO₃ (g) | 99 | 89 | 89 |
| Powder 2: Inorganic ZnO (g) | 0 | 10 | 10 |
| Metal salts/Nanoparticle | 1.6 | 1.6 | 1.6 |
| Precursor (g): Silver | | | |
| Nitrate (AgNO₃) | | | |
| Reducing agent (g) | 1 | 1 | 1 |
| Surfactant (g) | 0 | 0 | 2.5 |
| Water (ml) | 100 | 100 | 100 |
| Alcohol (ml) | 50 | 50 | 50 |
| Output Composition | 1%-99% | 1%-89%-10% | 1%-89%-10% |
| | Ag—CaCO₃ | Ag—CaCO₃—ZnO | Ag—CaCO₃—ZnO |

TABLE 2
Gram Negative Microbe: Salmonella enterica 10708

| | Contact | | | |
|---|---|---|---|---|
| | Time (hr.) | CFU/coupon | Effectiveness | Interpretation |
| Foam S-1 Control | 0 | 1.12E+05 | 0.00000 | Microbial Growth |
| (without nanohybrid) | 24 | 8.30E+06 | | |
| 010-702 nanohybrid- | 0 | 1.95E+05 | 91.66627 | Microbial Reduction |
| polyurethane foam | 24 | 7.00E+05 | | |
| 010-703 nanohybrid- | 0 | 1.82E+05 | 96.98795 | Microbial Reduction |
| polyurethane foam | 24 | 2.50E+05 | | |

TABLE 3

| | | Contact | | |
|---|---|---|---|---|
| Microorganism | Sample | Time (hr.) | CFU/Swatch | % Reduction |
| Staphylococcus | Control PA film | 0 | 7.20E+04 | No reduction |
| aureus | (no nanohybrid) | 24 | 1.10E+06 | |
| | Ag—Cu—SiO₂/PA | 0 | 7.95E+04 | 99.9874% |
| | nanocomposite | 24 | <1.00E+01 | |
| Salmonella | Control PA film | 0 | 1.31E+05 | No reduction |
| enterica | (no nanohybrid) | 24 | 2.52E+07 | |
| | Ag—Cu—SiO2/PA | 0 | 1.28E+05 | 99.9922% |
| | nanocomposite | 24 | <1.00E+01 | |

TABLE 4

| | Microbe- S. epidermidis | | | Microbe- Escherichia coli | | |
|---|---|---|---|---|---|---|
| | | | % | | | % |
| | | % | Reduction | | % | Reduction |
| | Surviving | Reduction | Against | Surviving | Reduction | Against |
| | CFU after | Against | Silver- | CFU after | Against | Silver- |
| Kevlar | 24 hours | Control | knitted | 24 hours | Control | knitted |
| Type | contact | Kevlar | Kevlar | contact | Kevlar | Kevlar |
| Control | 1.05E+04 | | | 7.23E+07 | | |
| (uncoated) | | | | | | |
| Silver | 3.43E+03 | | | 1.81E+03 | | |
| Knitted | | | | | | |
| Nanohybrid | <5 | 99.95% | 99.85% | <5 | 99.99% | 99.72% |
| coated | | | | | | |

TABLE 5

| | For Polyester Textiles |
|---|---|
| Polymeric Treatment Formulations | Antimicrobial + Hydrophobic |
| Aminofunctional nanohybrid (wt. %) | 2 |
| Wetting agent (vol. %) | 0.5 |
| Heat-reactive acrylic co-polymer (vol. %) | 1 |
| Hyperbranched polymer (vol. %) | 10 |
| (non-halogenated, APEO & fluorine-free) | |
| Blocked Isocyanate (vol. %) | 2 |
| Output Hydrophobicity (fluorocarbon-free) | ≤1.0 g |
| Liquid penetration of water by impact: | |
| AATCC 42 | |

TABLE 6

| | For Cotton Textiles | |
|---|---|---|
| | Antimicrobial | Antimicrobial + |
| Polymeric Treatment Formulations | only | Hydrophobic |
| Aminofunctional nanohybrid (wt. %) | 2 | 2 |
| Wetting agent (vol. %) | 0.5 | 0.5 |
| Heat-reactive acrylic co-polymer (vol. %) | 1 | 1 |
| C6- fluorocarbon polymer (vol. %) | 0 | 6 |
| (PFOA & PFOS-free) | | |
| Blocked Isocyanate (vol. %) | 0 | 2 |
| Output Hydrophobicity (fluorocarbon-based) | No repellency | ≤1.0 g |
| Liquid penetration of water by impact: | | |
| AATCC 42 | | |

We claim:

1. An antimicrobial nanohybrid structure synthesized by the following steps:
a. saturating a first carrier material with a plethora of metallic salt precursors thereby creating a mixture,
b. reacting the mixture with a reducing agent in the presence of a surface activated agent to reduce the plethora of metallic salts into a plethora of metallic nanoparticles and depositing the plethora of nanoparticles onto the first carrier material,
c. coating the surface of the resultant compound of nanoparticles and first carrier material with an organosilane coupling agent,
wherein the metallic salt precursor can be any hydrolyzable or water-soluble salt that can be chemically reduced to metallic nanoparticles with inherent antimicrobial properties.

2. The antimicrobial nanohybrid structure of claim 1 wherein the inherent antimicrobial properties include antimicrobial efficacy against gram-positive and gram-negative bacteria, fungi, and viruses is greater than 90%.

3. The antimicrobial nanohybrid structure of claim 2 wherein the metallic salt precursor is a material selected from the group consisting of silver, copper, copper oxide, zinc oxide, nickel, selenium, titanium, and titanium dioxide.

4. The antimicrobial nanohybrid structure of claim 2 wherein the coupling agent is an organosilane with an organofunctional group.

5. The antimicrobial nanohybrid structure of claim 2 wherein the coupling agent is an organosilane with an organofunctional group selected from the group consisting of a vinyl group, epoxy group, mercapto groups, amino groups, methacryloxy group, isocyanate groups, thiol groups, methoxy groups, and ethoxy groups.

6. The antimicrobial nanohybrid structure of claim 2 wherein the carrier material is selected from the group consisting of metal hydroxides, hydroxycarbonates, borates, inorganic phosphates, melamine, metal carbonates, hydrogen carbonates, aluminosilicates, silicate minerals, Al₂O₃, ZnO, TiO₂, MgO, CaO, ZrO₂, Cr₂O₃, SiO₂, Copper Oxide, Ag₂O, chromium, cobalt, nickel, and copper; titanium alloys, cobalt-chromium alloys, stainless steel, tantalum, MoS₂, graphite, hexagonal boron nitride, PTFE, calcium fluoride; graphene, graphene oxide, carbon black, charcoal, carbon nanotubes, benzalkonium chloride, silicon, silicon carbide, germanium, and gallium arsenide, zinc phosphate, zinc molybdate, aluminum phosphate; calcium sodium borosilicate, calcium aluminum borosilicate, carbamide peroxide, lysozyme, chitosan, starch, collagen peptides, lignin, nanocrystalline and nano-fibrillated cellulose.

7. An antimicrobial nanohybrid structure produced by the following steps:
a. combining antimicrobial particles, a first carrier material, and auxiliary additives to form a first mixture,
b. homogenizing the first mixture utilizing mechanochemical synthesis to yield a homogeneous second mixture of antimicrobial nanoparticles and first carrier material,
c. coating the surface of the second mixture of nanoparticles and first carrier materials with an organosilane coupling agent,
wherein the antimicrobial particles belongs to metallic species with inherent antimicrobial properties.

8. The antimicrobial nanohybrid structure of claim 7 wherein the inherent antimicrobial properties include antimicrobial efficacy against gram-positive and gram-negative bacteria, fungi, and viruses is greater than 90%.

9. The antimicrobial nanohybrid structure of claim 8 wherein the metallic salt precursor is a material selected from the group consisting of silver, copper, copper oxide, zinc oxide, nickel, selenium, titanium, and titanium dioxide.

10. The antimicrobial nanohybrid structure of claim 8 wherein the coupling agent is an organosilane with an organofunctional group.

11. The antimicrobial nanohybrid structure of claim 8 wherein the coupling agent is an organosilane with an organofunctional group selected from the group consisting of a vinyl group, epoxy group, mercapto groups, amino groups, methacryloxy group, isocyanate groups, thiol groups, methoxy groups, and ethoxy groups.

12. The antimicrobial nanohybrid structure of claim 8 wherein the carrier material is selected from the group consisting of metal hydroxides, hydroxycarbonates, borates, inorganic phosphates, melamine, metal carbonates, hydrogen carbonates, aluminosilicates, silicate minerals, Al₂O₃, ZnO, TiO₂, MgO, CaO, ZrO₂, Cr₂O₃, SiO₂, Copper Oxide, Ag₂O, chromium, cobalt, nickel, and copper; titanium alloys, cobalt-chromium alloys, stainless steel, tantalum, MoS₂, graphite, hexagonal boron nitride, PTFE, calcium fluoride; graphene, graphene oxide, carbon black, charcoal, carbon nanotubes, benzalkonium chloride, silicon, silicon carbide, germanium, and gallium arsenide, zinc phosphate, zinc molybdate, aluminum phosphate; calcium sodium borosilicate, calcium aluminum borosilicate, carbamide peroxide, lysozyme, chitosan, starch, collagen peptides, lignin, nanocrystalline and nano-fibrillated cellulose.

13. A antimicrobial polymer composition produced by the following steps:
a. producing antimicrobial nanohybrid structure; and
b. incorporating and reinforcing a polymer/co-polymer matrix with the antimicrobial nanohybrid structures by methods selected from the group of melt blending, shaping or forming, polymer solution casting and/or additive (3D) manufacturing of polymeric/plastic components.

14. The antimicrobial polymer composition of claim 13 wherein the polymers are selected from the group consisting of thermoplastic, thermosetting, and/or biopolymers category of polymers.

15. The antimicrobial polymer composition of claim 13 wherein the polymers are selected from the group consisting of polypropylene, polyethylene, polyamide, PVC, polycarbonate, ABS and PBT.

16. The antimicrobial polymer composition of claim 13 wherein the processes can be accomplished in solid, semi-solid, and liquid phases of matrix polymers.

17. The antimicrobial polymer composition of claim 13 wherein the composition is a solid selected from the group comprising fibers, wires, tubes, pellets, bottles, woven/non-woven fabrics, films, coated substrates.

18. The antimicrobial polymer composition of claim 13 wherein the composition is a liquid such as a polymer resin and coating formulation.

19. The antimicrobial polymer composition of claim 13 wherein the antimicrobial efficacy against gram-positive and gram-negative bacteria, fungi, and viruses is greater than 90%.
